(19) United States
(12) Patent Application Publication
(10) Pub. No.: US 2021/0007958 A1
(43) Pub. Date: Jan. 14, 2021

(54) SOLID COSMETIC CLEANSING AGENTS

(71) Applicant: Henkel AG & Co. KGaA, Duesseldorf (DE)

(72) Inventors: SOEREN SCHEELE, Pinneberg (DE); MANUELA METTE, Kleinfeld (DE); PETRA WESTPHAL, Neu Wulmstorf (DE); THOMAS SCHROEDER, Hamburg (DE)

(73) Assignee: Henkel AG & Co. KGaA, Duesseldorf (DE)

(21) Appl. No.: 16/917,390

(22) Filed: Jun. 30, 2020

(30) Foreign Application Priority Data
Jul. 10, 2019 (DE) 10 2019 210 158.5

(51) Int. Cl. A61K 8/46; A61K 8/34; A61K 8/36; A61K 8/73; A61K 8/02; A61Q 19/10; A61Q 5/02

(52) U.S. Cl. CPC A61K 8/466; A61K 8/345; A61K 8/361; A61Q 5/02; A61K 8/732; A61K 8/0216; A61Q 19/10; A61K 8/731

(57) ABSTRACT

The present disclosure concerns a solid cosmetic composition including—based on the total weight of the cosmetic composition—from about 15.0 to about 50.0% by weight of at least one surfactant, from about 10.0 to 60.0% by weight of at least one polyhydric C₂-C₆ alcohol, from about 0.1 to about 10.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and from about 0.1 to about 20% by weight of at least one polysaccharide selected from starch fractions and/or derivatives of starches, cellulose and/or cellulose derivatives, process for the preparation and packaging of corresponding cosmetic compositions, and methods of preparation and application and uses thereof.

### CROSS-REFERENCE TO RELATED APPLICATION

This application claims priority to German Patent Application No. 10 2019 210 158.5, filed Jul. 10, 2019, which is incorporated herein by reference in its entirety.

### TECHNICAL FIELD

The application describes solid cosmetic compositions based on surfactants, specific polyols, polysaccharides, fatty alcohols and/or fatty acid(s), in particular solid cleansing compositions which dissolve and foam in contact with water. The application further describes methods for the preparation of solid cosmetic compositions and their use for cleansing the human body, including the hair.

### BACKGROUND

Surfactant-containing products for the cleansing of the human body and hair have been known for a long time and are offered commercially mainly as foam baths, shower preparations, shampoos and/or oral care compositions in liquid or paste form in suitable packaging. End consumers take the necessary amount of product from the packaging during use and dispose of it after emptying. Compared to classical soaps that are sold in solid form, such products offer the user the advantage of easy and quick handling, which is why they dominate the market today. However, this advantage is achieved by accepting certain disadvantages, which will be discussed below. In most cases, the packaging of the described products in liquid or pasty form includes non-recyclable plastic, which is a serious problem from an environmental point of view in view of the constantly growing amount of plastic waste.

A further problem is that previous products usually contained higher quantities of water or water/solvent mixtures, which means that the products have a larger volume and, possibly of greater importance, from a transport point of view, a relatively high weight. This is disadvantageous for several reasons. In times of increasing water scarcity, resources should be saved. Also important from both an environmental and cost perspective is an undesirable, increased transport volume associated with large-volume heavy products. Another interesting point is that worldwide travel activity is constantly increasing. Consumers are therefore increasingly interested in cosmetic products that are easy to transport due to their low weight and volume. This is particularly relevant with regard to air travel, as larger containers containing liquids are generally excluded from being carried in an aircraft cabin, so that a passenger travelling with only hand luggage often finds himself in the situation, due to the cosmetics products dominating the market today, of not being able to take his preferred product selection with him or of having to decant the respective products into smaller containers first, which, however, is generally accompanied by an even higher volume of packaging material.

The provision of alternative product forms with lower water content, contained in more environmentally friendly, e.g. recyclable, packaging in a space-saving manner, is therefore an important objective in the formulation of improved, contemporary and sustainable cosmetic products.

Classic bars of soap have been known and used for a long time. They also generally have a very low water or solvent content and are often packaged with little material, but their handling is not pleasant for many people because a started bar of soap is difficult to transport, often partially dissolves when lying next to the sink, which is also inefficient, and makes the sink look unattractive due to soap residues and because bars of soap have a tendency to slip out of the user's hand when wet.

Furthermore, classic soap formulations are only conditionally suitable for regular cleansing of the entire human body. Due to their skin drying potential they are mainly used for cleansing the hands which are more tolerant in this respect than other skin areas. Furthermore, bars of soap are controversial from a hygiene point of view because of their multiple uses, especially if they are used by several end users.

A further disadvantage of conventional soap formulations, especially with rather small bars of soap, is that it takes some time for enough of the bar of soap to come off to achieve the desired amount of lather and the desired cleansing effect. On the one hand, this is usually not desired by users because of the additional time required, and on the other hand, it may be associated with higher water consumption for personal hygiene, as many users do not turn off the water flow of the shower or tap when soaping. From these aspects it does not make sense to market single application portions of classic soap formulations, especially in miniaturized form of a classic bar of soap as their dissolution is too slow as the formulations of these soaps are not optimized for the cleansing of larger skin areas or the cleansing of hair.

### BRIEF SUMMARY

Solid cosmetic compositions and methods of preparing the same are provided herein. In an embodiment, a solid cosmetic composition includes—based on the total weight of the cosmetic composition—
a) from about 15.0 to about 50.0% by weight of at least one surfactant,
b) from about 10.0 to about 60.0% by weight of at least one polyhydric C₂-C₆ alcohol,
c) from about 0.1 to about 10.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) from about 0.1 to about 20% by weight of at least one polysaccharide selected from
starch fractions and/or derivatives of starches, and/or
cellulose and/or cellulose derivatives.

In another embodiment, a solid cosmetic composition includes—based on the total weight of the cosmetic composition—
a) from about 25.0 to about 40.0% by weight of at least one surfactant,
b) from about 30.0 to about 60% by weight of glycerol,
c) from about 3.0 to about 10% by weight of at least one saturated or unsaturated, branched or unbranched C₁₀-C₂₂ carboxylic acid and/or their salts, and
d) from about 0.1 to about 20% by weight of at least one polysaccharide selected from
   starch fractions from corn, potatoes, rice, wheat and/or tapioca, and/or derivatives thereof, and/or
   cellulose and/or cellulose derivatives.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

In view of the problems and requirements described above, formulations and manufacturing processes suitable for solid cosmetic cleansing agents are provided which, by their nature, are suitable for packaging in individual application portions, as well as methods for the manufacture of the same and uses thereof. Several measures have been established which may contribute to this suitability. Thus, the task of the present disclosure is solved by the formulation of methods and uses described in detail below:

A solid cosmetic composition including—based on the total weight of the cosmetic composition—
a) from about 15.0 to about 50.0% by weight of at least one surfactant,
b) from about 1510.0 to about 60.0% by weight of at least one polyhydric C₂-C₆ alcohol,
c) from about 150.1 to about 10.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) from about 150.1 to about 20% by weight of at least one polysaccharide selected from
   starch fractions and/or derivatives of starches,
   cellulose and/or cellulose derivatives.

A solid cosmetic composition as contemplated herein, including at least one anionic surfactant a).

A solid cosmetic composition as contemplated herein, comprising—based on the total weight of the cosmetic composition—from about 17.5 to about 47.5% by weight, preferably from about 20.0 to about 45.0% by weight, more preferably from about 22.5 to about 42.5% by weight and most preferably from about 25.0 to about 40.0% by weight of at least one surfactant a), preferably an anionic surfactant.

A solid cosmetic composition as contemplated herein, comprising
ai) at least one anionic isethionate surfactant of the formula (I)

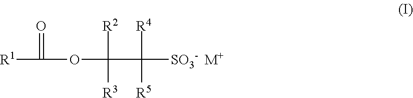

wherein
   R¹ is a linear or branched, saturated or unsaturated alkyl radical having from about 6 to about 30 carbon atoms,
   R² to R⁵ independently of one another represent a hydrogen atom, a C₁-C₄ alkyl radical or a C₁-C₄ hydroxyalkyl radical, and/or
   M⁺ represents an ammonium, an alkanolammonium or a metal cation and/or
aii) at least one anionic taurate surfactant of formula (II)

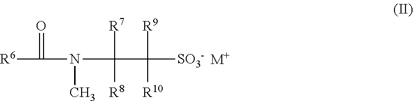

wherein
   R⁶ represents a linear or branched, saturated or unsaturated alkyl radical having from about 6 to about 30 carbon atoms,
R⁷ to R¹⁰ independently of one another represent a hydrogen atom, a C₁-C₄ alkyl radical or a C₁-C⁴ hydroxyalkyl radical, and
M⁺ stands for an ammonium, an alkanol ammonium or a metal cation.

A solid cosmetic composition as contemplated herein, comprising at least one anionic isethionate surfactant of formula (I) and at least one anionic taurate surfactant of formula (II).

A solid cosmetic composition as contemplated herein, comprising (relative to the total weight of the composition)
i. from about 10.0 to about 40% by weight, preferably from about 12.5 to about 35% by weight, particularly preferably from about 15.0 to about 32.5% by weight and in particular from about 17.5 to about 30.0% by weight of at least one anionic isethionate surfactant according to formula (I), and
ii. from about 1.0 to about 10% by weight, preferably from about 2.0 to about 9.0% by weight, particularly preferably from about 2.5 to about 8.0% by weight and in particular from about 3.0 to about 7.0% by weight of at least one anionic taurate surfactant corresponding to formula (II).

A solid cosmetic composition as contemplated herein, comprising at least one C₂-C₆ polyhydric alcohol b) in an amount of from about 15.0% to about 60% by weight, preferably from about 20.0% to about 60% by weight, more preferably from about 25.0% to about 60% by weight, most preferably from about 25.0% to about 60% by weight and more particularly from about 30.0% to about 60% by weight (relative to the total weight of the composition).

A solid cosmetic composition as contemplated herein, including as at least one polyhydric alcohol b) 1,2-propylene glycol, 1,3-butylene glycol, dipropylene glycol, glycerol and/or diglycerol.

A solid cosmetic composition as contemplated herein, including as polyhydric alcohol b) glycerol.

A solid cosmetic composition as contemplated herein, comprising at least one component c) in an amount of from about 0.5 to about 10% by weight, preferably from about 1.0 to about 10% by weight, more preferably from about 2.0 to about 10% by weight and most preferably from about 3.0 to about 10% by weight (based on the total weight of the composition).

A solid cosmetic composition as contemplated herein, comprising as at least one component c) saturated or unsaturated, branched or unbranched C₈-C₃₀ carboxylic acids and/or their salts, preferably C₁₀-C₂₂ carboxylic acids and/or their salts and in particular coco acids, lauric acid, myristic acid, palmitic acid, stearic acid, behenic acid, oleic acid and mixtures thereof and/or the salts of these acids.

A solid cosmetic composition as contemplated herein, comprising coco acids, stearic acid and/or the salts of these acids, in particular coco acids and sodium stearate.

A solid cosmetic composition as contemplated herein, comprising at least one component d) in an amount of from about 0.5 to about 17.5% by weight, preferably from about 1.0 to about 15.0% by weight, more preferably from about 1.5 to about 12.5% by weight and most preferably from about 2.0 to about 10.0% by weight (based on the total weight of the composition).

A solid cosmetic composition as contemplated herein, including as at least one polysaccharide d)
i. starch fractions from maize, potatoes, rice, wheat and/or tapioca, and/or derivatives of starches such as amylose, amylopectin, dextrin, and/or
ii. cellulose and/or cellulose derivatives such as alkyl cellulose, carboxyalkyl cellulose, hydroxyalkyl cellulose.

A solid cosmetic composition as contemplated herein, including as at least one polysaccharide d) one or more ingredients from group i.

A solid cosmetic composition as contemplated herein, including—based on the total weight of the composition—
from about 1.0 to about 15% by weight of maize starch and
from about 0.1 to about 10% by weight of dextrins, preferably maltodextrin.

A solid cosmetic composition as contemplated herein, including, relative to the total weight of the cosmetic composition, from about 0.01 to about 5.00% by weight of at least one cationic polymer e), preferably at least one cationic polysaccharide polymer obtainable from guar, cassia and/or inulin.

A solid cosmetic composition as contemplated herein, comprising at least one cationic polymer known by the INCI name Hydroxypropyl Trimonium Inulin.

A solid cosmetic composition as contemplated herein, including—based on the total weight of the cosmetic composition—from about 0.01 to about 5.00% by weight of at least one oil, fat and/or wax component f), preferably a naturally occurring oil, fat or wax.

A solid cosmetic composition as contemplated herein, including apricot kernel oil, argan oil, jojoba oil, marula oil, almond oil, olive oil, coconut oil and/or sunflower oil.

A solid cosmetic composition as contemplated herein, including—based on the total weight of the cosmetic composition—from about 0.001 to about 0.01% by weight of at least one bitter substance g), preferably a compound known under the INCI designation Denatonium Benzoate.

A solid cosmetic composition as contemplated herein for the cleansing and/or care of human skin and/or human hair.

A solid cosmetic composition as contemplated herein including water in an amount of up to about 15% by weight, preferably about 12.5% by weight and in particular about 10% by weight (based on the weight of the composition).

A solid cosmetic composition as contemplated herein in the form of a powder which dissolves and foams in contact with water.

A solid cosmetic composition as contemplated herein in the form of a pressed or shaped body which has a density in the range from about 0.6 g/cm³ to about 1.1 g/cm³ and which dissolves and foams in contact with water.

A solid cosmetic composition as contemplated herein in the form of a porous body which has a density in the range from about 0.2 g/cm³ to about 0.9 g/cm³ and which dissolves and foams in contact with water.

A method for preparing a solid cosmetic composition as contemplated herein, wherein all the ingredients are mixed and optionally sieved in a mixing device for cosmetic powder products.

A method for preparing a solid cosmetic composition as contemplated herein, exemplified by the following steps:
a. mixing all ingredients in a mixing device for cosmetic powder products,
b. converting the mixture into a suitable form, and
c. pressing the mixture into the desired shape.

A method for preparing a solid cosmetic composition as contemplated herein, exemplified by the following steps:
d. adding all ingredients into a heatable container,
e. heating the mixture a) at a temperature of up to about 75° C. until the ingredients are mixed,
f. extruding the mixture a) from a desired shaping port.

A method for preparing a solid cosmetic composition as contemplated herein, exemplified by the following steps:
g. adding all ingredients into a heatable container,
h. heating the mixture a) at a temperature of up to about 75° C. until the ingredients are mixed,
i. introducing a gas by
   i. introducing air, N₂, N₂O and/or CO₂ at a pressure of from about 2 to about 40 bar; or
   ii. introducing air with a high speed mixer,
j. extruding the aerated mixture c) from a desired shaping port or into a desired shape,
k. solidification/cooling of the extrudate in the desired form.

Solid cosmetic cleansing compositions obtainable by methods as contemplated herein.

A method of cleansing the human body including the hair, wherein a solid cosmetic composition is moistened with water or soaked in water or dissolved in water, rubbed between the hands and/or foamed, spread over the part of the body to be cleansed and rinsed with water as contemplated herein.

A method of applying a solid cosmetic composition as contemplated herein, in wherein
a) the solid composition is first mixed with water and then applied to the hair and worked into it, or
b) the solid composition is applied directly to the wet hair and worked in.

The use of a solid cleansing composition as contemplated herein for cleansing and/or care of the human body.

The first subject-matter of this application is a solid cosmetic composition including—relative to the total weight of the cosmetic composition—
a) from about 15.0 to about 50.0% by weight of at least one surfactant,
b) from about 10.0 to about 60.0% by weight of at least one polyhydric C₂-C₆ alcohol,
c) from about 0.1 to about 10.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) from about 0.1 to about 20% by weight of at least one polysaccharide selected from
   starch fractions and/or derivatives of starches,
   cellulose and/or cellulose derivatives.

The compositions as contemplated herein are solid at 25° C. Solid compositions within the meaning of the present application are three-dimensional, dimensionally stable structures which are not liquid or gaseous, i.e. which maintain their outer shape even without a surrounding vessel. However, the term “solid” does not imply anything about density or elasticity or other physical properties, so that jellies, brawn, butter etc. may also be solid as contemplated herein as long as they are dimensionally stable at 25° C.

Solid compositions within the meaning of the present application may also be powders.

The previously defined formulations are solid in this sense and offer the right properties for single application portions, especially with regard to their dissolving and foaming behavior during use. The high concentrations of the active ingredients in such a composition are associated with the advantages that few resources are consumed during production and transport and that the products are easy to transport without great effort or restrictions, even after they have reached the hands of a consumer via the trade, whether to the gym or on a flight.

The solid cosmetic composition described above may include as at least one surfactant a) surfactant(s) from the group of anionic, amphoteric, zwitterionic or non-ionic surfactants or a mixture thereof.

In a preferred embodiment, the compositions as contemplated herein include—based on their total weight—from about 17.5 to about 47.5% by weight, preferably from about 20.0 to about 45.0% by weight, particularly preferably from about 22.5 to about 42.5% by weight and in particular from about 25.0 to about 40.0% by weight of at least one anionic surfactant a).

Anionic surfactants carry a negative charge in their hydrophilic part. Chemically they are generally salts of sodium, potassium, ammonium, magnesium, monoethanolamine, diethanolamine or triethanolamine.

Among the basically suitable anionic surfactant types that may be used in the agents as contemplated herein are, for example:
linear and branched fatty acids with from about 8 to about 30 C atoms (soaps),
ether carboxylic acids of the formula R—O—(CH₂—CH₂O)ₓ—CH₂—COOH, wherein R is a linear or branched, saturated or unsaturated alkyl group having from about 8 to about 30 C atoms and x=0 or 1 to 16,
acyl sarcosines with from about 8 to about 24 C atoms in the acyl group (sarcosinate surfactants),
acyl taurines with from about 8 to about 24 C atoms in the acyl group (taurate surfactants),
acyl isethionates with from about 8 to about 24 C atoms in the acyl group (isethionate surfactants),
sulphosuccinic acid mono and/or dialkyl esters with from about 8 to about 24 C atoms in the alkyl group and sulphosuccinic acid mono alkyl polyoxyethylene esters with from about 8 to about 24 C atoms in the alkyl group and from 1 to 6 oxyethyl groups (sulfosuccinate surfactants),
alpha-olefin sulfonates with from about 8 to about 24 C atoms (alpha-olefin sulphonate surfactants),
alkyl sulfates and/or alkyl ether sulfate salts of the formula R—(OCH₂—CH₂)ₙ—O—SO₃X, wherein R preferably denotes a straight-chain or branched, saturated or unsaturated alkyl group having from about 8 to about 30 C atoms, x denotes the number 0 or 1 to 12 and X denotes an alkali metal, alkaline earth metal, ammonium or alkanolamine ion,
sulfonates of unsaturated fatty acids with from about 8 to about 24 C atoms and from 1 to 6 double bonds,
esters of tartaric acid and citric acid with alcohols which are products of the addition of about 2-15 molecules of ethylene oxide and/or propylene oxide to fatty alcohols with 8 to 22 carbon atoms, and/or
alkyl and/or alkenyl ether phosphates of the formula

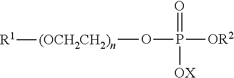

wherein R¹ is preferably an aliphatic hydrocarbon radical having from about 8 to about 30 carbon atoms, R² is hydrogen, a radical (CH₂CH₂O)ₙR¹ or X, n stands for numbers from 0 to about 10 and X for hydrogen, an alkali metal or alkaline earth metal or the group —NR³R⁴R⁵R⁶, with R³ to R⁶ independently of one another standing for a C₁-C₄ hydrocarbon radical.

Amino acid-based surfactants are particularly suitable in the light of the present disclosure because they have excellent skin compatibility and are available from natural sources.

Due to their very good incorporation into solid compositions in terms of the present disclosure, isethionate and/or taurate surfactants have proven to be particularly suitable.

The present disclosure therefore further concerns a solid cosmetic composition as described above, including
i. at least one anionic isethionate surfactant of formula (I)

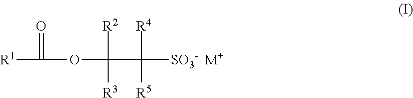

wherein
   R¹ is a linear or branched, saturated or unsaturated alkyl radical having from about 6 to about 30 carbon atoms,
   R² to R⁵ independently of one another represent a hydrogen atom, a C₁-C₄ alkyl radical or a C₁-C₄ hydroxyalkyl radical, and/or
   M⁺ represents an ammonium, an alkanolammonium or a metal cation and/or
ii. at least one anionic taurate surfactant of formula (II)

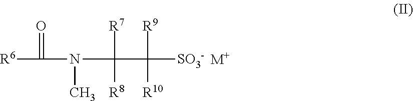

wherein
   R⁶ is a linear or branched, saturated or unsaturated alkyl radical having from about 6 to about 30 carbon atoms,
   R⁷ to le independently of one another represent a hydrogen atom, a C₁-C₄ alkyl radical or a C₁-C₄ hydroxyalkyl radical, and
   M⁺ represents an ammonium, an alkanolammonium or a metal cation.

Anionic isethionate and taurate surfactants are mild, anionic surfactants, ideal for the manufacture of shampoos, shampoo bars, body washes, liquid soaps, facial care products and bath bombs. They make hair easy to comb, smooth, soft and shiny. They may be produced on a purely vegetable basis and may therefore also be used for vegan cosmetics. A raw material source may be fatty acids of coconut oil.

In a particularly preferred embodiment, the compositions as contemplated herein include at least one anionic isethionate surfactant according to formula (I) and at least one anionic taurate surfactant according to formula (II).

The present disclosure further concerns a solid cosmetic composition as described above, including—based on the total weight of the cosmetic composition—
i. from about 10.0 to about 40% by weight, preferably from about 12.5 to about 35% by weight, particularly preferably from about 15.0 to about 32.5% by weight and in particular from about 17.5 to about 30.0% by weight of at least one anionic isethionate surfactant corresponding to formula (I), and
ii. from about 1.0 to about 10% by weight, preferably from about 2.0 to about 9.0% by weight, particularly preferably from about 2.5 to about 8.0% by weight and in particular from about 3.0 to about 7.0% by weight of at least one anionic taurate surfactant corresponding to formula (II).

These concentration ranges have proved to be particularly suitable. When working with these concentration ranges, the solid cosmetic compositions obtained with them have balanced properties in terms of firmness, solubility behavior, application feel and cleansing performance.

Suitable amphoteric and/or zwitterionic surfactant types which may be used in the compositions as contemplated herein include, for example, one or more compounds of the following formulae (i) to (vii). Here the radical R preferably represents a straight-chain or branched, saturated or mono- or polyunsaturated alkyl or alkenyl radical having from about 7 to about 23 carbon atoms (formulae (i) and (ii)) or a straight-chain or branched, saturated or mono- or polyunsaturated alkyl or alkenyl radical having from about 8 to about 24 carbon atoms (formulae (iii) to (vii)):

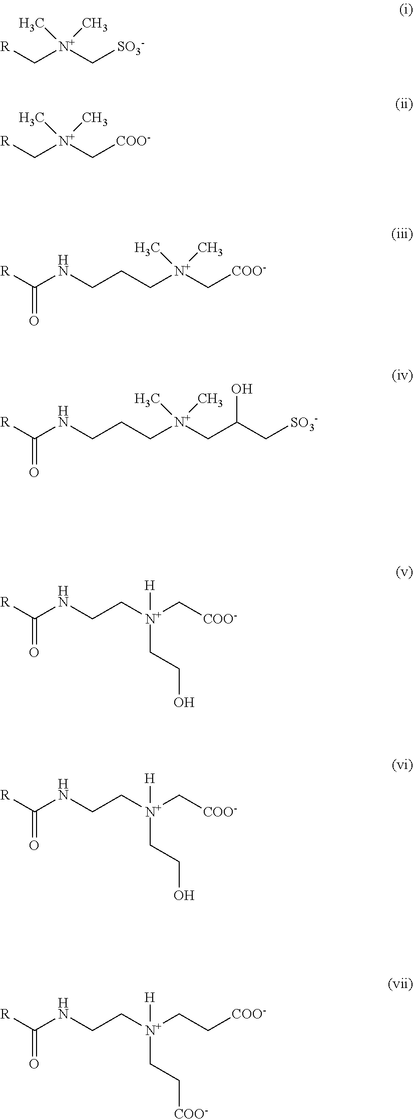

Preferred amphoteric surfactants of the above-mentioned formulae (i) to (vii) include as residue R predominantly a straight-chain or branched, saturated, mono- or polyunsaturated alkyl radical with from about 8 to about 20, more preferably from about 8 to about 18 and in particular with from about 8 to about 16 C atoms. Amphoteric surfactants wherein the radical R is derived from coconut oil are particularly preferred.

Particularly preferred are those under the INCI designations Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaines, Lauryl Betaine Cocamidopropyl Betaine and/or Lauramidopropyl Betaine are known and commercially available amphoteric surfactants from several suppliers.

Surfactants with the INCI designations Cocamidopropyl Betaine, Lauramidopropyl Betaine, Cocoampho(di)acetates and/or Lauroampho(di)acetates are particularly preferred.

The amphoteric and/or zwitterionic co-surfactant(s) may be used in the compositions as contemplated herein (based on their total weight) preferably in an amount from 0 to about 10.0% by weight, more preferably from about 0.1 to about 8.0% by weight, particularly preferably from about 0.5 to about 6.0% by weight and in particular from about 0.75 to about 5.00% by weight.

Suitable non-ionic surfactants for use as co-surfactants are for example
Amine oxides, which may be selected from compounds of the general formulae (I) or (II)

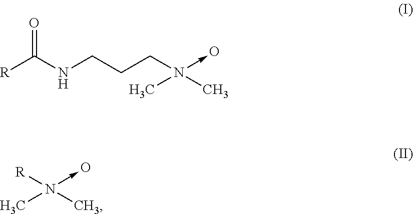

wherein R in each case represents a straight-chain or branched, saturated or mono- or polyunsaturated alkyl or alkenyl radical having 6 to 24 carbon atoms, preferably having 8 to 18 carbon atoms.
Particularly preferred are the surfactants of the above-mentioned formulae (I) or (II) known under the INCI designations Cocamine Oxide, Lauramine Oxide and/or Cocamidopropylamine Oxide and commercially available from various suppliers.
Fatty acid alkanolamines corresponding to the following general formula,

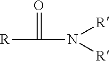

wherein R preferably denotes a linear or branched, saturated or unsaturated alkyl or alkenyl group including from about 8 to about 24 carbon atoms and the groups radical R′ represent hydrogen or the group —(CH₂)ₙOH, wherein n denotes the numbers 2 or 3, with the proviso that at least one of the groups radical R′ represents the abovementioned group —(CH₂)ₙOH,
fatty alcohol alkoxylates (especially ethoxylates) with a C-chain length of from about 8 to about 24, particularly from about 10 to about 20, and a degree of alkoxylation (degree of ethoxylation) of from 2 to about 30, such as laureth-4,
mixtures of alkyl (oligo) glucosides and fatty alcohols, for example the commercially available product Montanov®68,
sterols. Sterols are a group of steroids that carry a hydroxyl group on C atom 3 of the steroid skeleton and are isolated from animal tissue (zoosterols) as well as from vegetable fats (phytosterols). Examples of zoosterols are cholesterol and lanosterol. Examples of suitable phytosterols are ergosterol, stigmasterol and sitosterol. Sterols, the so-called mycosterols, are also isolated from fungi and yeasts.
Phospholipids. These are mainly the glucose phospholipids, which are obtained as lecithins or phosphatidylcholines from e.g. egg yolks or plant seeds (e.g. soybeans),
alkyl (oligo) glycosides. Suitable alkyl (oligo) glycosides may be selected from compounds of the general formula RO-[G]ₓ, wherein [G] is preferably derived from aldoses and/or ketoses having 5-6 carbon atoms, preferably from glucose.
The index number x represents the degree of oligomerization (DP), i.e. the distribution of the mono- and oligoglycosides. The index number x preferably has a value in the range from 1 to about 10, particularly preferably in the range from 1 to 3, whereby it may not be an integer but a fractional number which may be determined analytically.
Particularly preferred alkyl (oligo) glycosides have a degree of oligomerization between 1.2 and 1.5. The radical R preferably represents at least one alkyl and/or alkenyl radical with from about 4 to about 24 C atoms.
Alkyl (oligo) glycosides which are particularly preferred are the compounds known under the INCI designations Caprylyl/Caprylic Glucosides, Decyl Glucosides, Lauryl Glucosides and Coco Glucosides.

Particularly preferred non-ionic surfactants, which may be contained in the agents as contemplated herein as co-surfactants, are fatty acid alkanolamines, alkyl (oligo) glucosides and amine oxides. Fatty acid alkanolamines are particularly preferred.

The non-ionic co-surfactant(s) may be used in the cosmetic compositions as contemplated herein (based on their total weight) preferably in an amount from 0 to about 3.0% by weight, more preferably from about 0.1 to about 2.50% by weight, particularly preferably from about 0.2 to about 2.25% by weight and in particular from about 0.25 to about 2.00% by weight.

The compositions as contemplated herein may, in addition to cleansing agents, also be conditioning agents. Conditioning agents suitable as contemplated herein may preferably be
at least one cationic surfactant, preferably in a total amount of from about 0.1 to about 10% by weight, more preferably from about 0.2 to about 9% by weight and particularly preferably from about 0.3 to about 8% by weight, each based on the weight of the composition, and
include at least one further surfactant selected from anionic, amphoteric, zwitterionic and/or non-ionic surfactants, preferably in a total amount of from about 0.1 to about 20% by weight, more preferably from about 0.2 to about 15% by weight and particularly preferably from about 0.3 to about 10% by weight, in each case based on the weight of the composition.

In a preferred embodiment, the compositions as contemplated herein include—based on their total weight—from about 15.0 to about 60% by weight, preferably from about 20.0 to about 60% by weight, particularly preferably from about 25.0 to about 60% by weight and in particular from about 30.0 to about 60% by weight of at least one polyhydric C₂-C₆ alcohol b).

Suitable polyhydric alcohols (b) include, for example, alditols such as mannitol, isomalt, lactitol, sorbitol and xylitol, threitol, erythritol and arabitol, 1,2-propylene glycol, 1,3-butylene glycol, dipropylene glycol, glycerol and/or diglycerol. Preferred are 1,2-propylene glycol, 1,3-butylene glycol, dipropylene glycol, glycerol and/or diglycerol. Glycerol is particularly preferred.

These polyhydric alcohols are well tolerated by the skin and as solvents they ensure that the solid cosmetic compositions available with them are not too solid or too difficult or slow to dissolve.

In a preferred embodiment, the compositions as contemplated herein include—based on their total weight—from about 0.5 to about 10% by weight, preferably from about 1.0 to about 10% by weight, particularly preferably from about 2.0 to about 10% by weight and in particular from about 3.0 to about 10% by weight of at least one component c).

Suitable components c) in the context of the present disclosure are saturated or unsaturated, branched or unbranched C₈-C₃₀ carboxylic acids and/or their salts, preferably C₁₀-C₂₂ carboxylic acids and/or their salts and in particular coco acids, lauric acid, myristic acid, palmitic acid, stearic acid, behenic acid, oleic acid and mixtures thereof and/or the salts of these acids. Particularly suitable are coco acids, stearic acid and/or the salts of these acids, in particular coco acids and sodium stearates.

These compounds have proved to be particularly suitable structure-giving ingredients for the purposes of the present disclosure. They may be used to formulate cosmetic compositions of sufficient strength which do not melt too low.

In a preferred embodiment, the compositions as contemplated herein include—based on their total weight—from about 0.5 to about 17.5% by weight, preferably from about 1.0 to about 15% by weight, particularly preferably from about 1.5 to about 12.5% by weight and in particular from about 2.0 to about 10% by weight of at least one component d).

Polysaccharides d) suitable for present disclosure are usually composed of more than ten monosaccharide units. Preferred polysaccharides are the starches composed of α-D-glucose units as well as starch degradation products such as amylose, amylopectin and dextrins. As contemplated herein, chemically and/or thermally modified starches are particularly advantageous, e.g. hydroxypropyl starch phosphate, dihydroxypropyl distarch phosphate or the commercial products Dry Flo®. Dextrans and their derivatives, e.g. dextran sulfate, are also preferred. Also preferred are non-ionic cellulose derivatives such as methyl cellulose, hydroxypropyl cellulose, hydroxypropyl methyl cellulose or hydroxyethyl cellulose. Other preferred examples are polysaccharides from fucose units, e.g. the commercial product Fucogel®.

The present disclosure also relates to a solid cosmetic composition as described above, including as polysaccharide d)
i. starch fractions from corn, potatoes, rice, wheat and/or tapioca, and/or derivatives of starches such as amylose, amylopectin, dextrin, and/or
ii. cellulose and/or cellulose derivatives such as alkyl cellulose, carboxyalkyl cellulose, hydroxyalkyl cellulose.

Particularly preferably, the compositions as contemplated herein include at least one polysaccharide d) from the aforementioned group i.

In particular preferably, the solid cosmetic compositions include (relative to their total weight)
from about 1.0 to about 15% by weight of maize starch and
from about 0.1 to about 10% by weight of dextrins, preferably maltodextrin.

These polysaccharides have proved to be particularly suitable stabilizing agents in the context of the present disclosure claimed here. Their use makes it possible to provide ready-made consumer products which retain their properties and appearance in a particularly stable manner over a long period of time and under various environmental conditions.

The present disclosure also relates to a solid cosmetic composition as described above, including—based on the total weight of the cosmetic composition—from about 0.01 to about 5.00% by weight of at least one cationic polymer e).

Cationic polymers have conditioning properties, i.e. they provide a pleasant skin or hair feel, thus offering added value. They may be used in the context of the present disclosure without significantly impairing the cleansing performance.

Particularly suitable are:
quaternized cellulose polymers, especially polyquaternium-10, as commercially available under the names Celquat® and Polymer JR®,
hydrophobically modified cellulose derivatives, such as the cationic polymers marketed under the trade name SoftCat®,
cationic alkyl polyglycosides,
cationized honey, for example the commercial product Honeyquat® 50,
cationic polysaccharide polymers from natural sources such as guar, cassia and/or inulin,
polymeric diallyl dimethyl ammonium salts and their copolymers with esters and amides of acrylic acid and methacrylic acid, mainly polyquaternium-6 and polyquaternium-7, commercially available under the names Merquat °100 (poly(diallyl dimethyl ammonium chloride)) and Merquat®550 (diallyl dimethyl ammonium chloride-acrylamide copolymer) are examples of such cationic polymers,
copolymers of vinylpyrrolidone with quaternized derivatives of dialkylaminoalkyl acrylate and methacrylate, such as diethyl sulfate-quaternized vinylpyrrolidone dimethylaminoethyl methacrylate copolymers. Such compounds are commercially available under the names Gafquat®734 and Gafquat®755,
vinylpyrrolidone vinylimidazolium methochloride copolymers, as offered under the names Luviquat® FC 370, FC 550, FC 905 and HM 552,
quaternized polyvinyl alcohol,
and the polymers known as polyquaternium-2, polyquaternium-17, polyquaternium-18, polyquaternium-24, polyquaternium-27, polyquaternium-32, polyquaternium-37, polyquaternium-74 and polyquaternium-89.

Cationic polysaccharide polymers are particularly preferred in the context of the present disclosure, which are obtained from natural sources (in particular from guar, cassia and/or inulin).

A content of these specific polymer types in the composition as contemplated herein is not only beneficial for improving the care properties for skin and hair, but it was also found that polymers in combination with other cationic polymers do not cause an over-conditioning effect even after regular application on hair.

Inulin is a polysaccharide which belongs to the group of fructans. In addition to a terminal glucose building block, it comprises up to about 60 fructose monomers in the chain, each of which is linked via β-2,1-glycosidic bonds. Inulin may be obtained from the leaves, roots, fruits and/or flowers of composites and/or umbellifers, such as Jerusalem artichokes, chicory, artichokes and/or parsnips.

Cationic inulin polymers particularly suitable as contemplated herein are cationically modified by reacting hydroxyl groups of the fructose building blocks with reactive quaternary ammonium compounds. The suitable quaternary ammonium compounds are preferably compounds of the following formula

N⁺(R¹R²R³R⁴)X⁻

wherein R¹, R² and R³ are methyl or ethyl groups and R⁴ is an epoxy-R⁵ or a halohydrin group Y—CH₂—CH(OH)—R⁵—, wherein R⁵ is a C₁-C₃ alkylene group, Y is a halide and X is an anion such as Cl−, Br−, I− or HSO4−. Particularly suitable cationic inulin polymers b) within the context of the present disclosure correspond to the formula

R—O—CH₂—CH(OH)—R⁵—N⁺(R¹R²R³)X⁻,

wherein R is inulin and the other radicals have the same meaning as above.

In a particularly preferred embodiment, the compositions as contemplated herein include cationic inulin polymers cationically modified with cationic hydroxy-C1-C3-alkyl-trialkylammonium groups, in particular with hydroxypropyl trimethyl ammonium groups. Within this embodiment, cationic inulin polymers known and commercially available under the INCI designation Hydroxypropyltrimonium Inulin are preferred.

The degree of cationic substitution of the cationic inulin polymers, in particular of cationic inulins known under the INCI designation Hydroxypropyltrimonium Inulin, may be varied and adjusted as required. For the use in cosmetic compositions as contemplated herein, it has proven to be particularly preferred if such cationically modified inulin polymers exhibit a higher degree of cationic modification (higher degree of cationic substitution), as this ultimately allows a better skin care performance to be achieved.

In a particularly preferred embodiment, the cationic inulin polymer contained in the cosmetic compositions as contemplated herein has a cationic charge density>1.5 meq/g, more preferably >2.0 meq/g, particularly preferably >2.5 meq/g, very preferably >3.0 meq/g and in particular >3.5 meq/g. Within this version it is particularly preferred if cationic inulin polymers known under the INCI designation Hydroxypropyltrimonium Inulin b) have a cationic charge density>1.5 meq/g, more preferably >2.0 meq/g, particularly preferably >2.5 meq/g, very preferably >3.0 meq/g and in particular >3.5 meq/g.

In a further particularly preferred embodiment, the cationic inulin polymer b) contained in the cosmetic compositions as contemplated herein has an average molar mass of from about 2,000 to about 50,000 g/mol, more preferably from about 2,500 to about 40,000 g/mol, particularly preferably from about 3,000 to about 30,000 g/mol, very preferably from about 3,500 to about 20,000 g/mol and in particular from about 4,000 to about 10,000 g/mol. Within this embodiment, it is particularly preferred if cationic inulin polymers known under the INCI designation Hydroxypropyltrimonium Inulin b) have an average molar mass of from about 2,000 to about 50,000 g/mol, more preferably from about 2,500 to about 40,000 g/mol, particularly preferably from about 3,000 to about 30,000 g/mol, very preferably from about 3,500 to about 20,000 g/mol and in particular from about 4,000 to about 10,000 g/mol.

It has been discovered that the combination of cationic inulin polymers and specific cationic polymers available from natural sources is particularly suitable for achieving very good hair care effects. Preferably, the cationic polymers derived from natural sources are cationic polygalactomannan derivatives.

Galactomannans are polysaccharides including combinations of mannose and galactose monomers in different contents. In these, the mannose units are linked to each other via β(1-4) glycosidic bonds; the galactose units are linked via α(1-6) bonds. The ratio of mannose to galactose monomers varies according to the type and origin of the plant and the temperature at which it is grown. In Greek fenugreek (fenugreek gum), the mannose-galactose ratio is about 1:1 (corresponding to one monomer of mannose to one monomer of galactose); in guar gum about 2:1; in tara gum about 3:1; in locust bean gum about 4:1 and in cassia gum about 5:1. All galactomannans from these sources are suitable for cationic modification and use as polymers in cosmetic compositions as contemplated herein. Guar gum and/or cassia gum are particularly suitable for use in the cosmetic compositions as contemplated herein.

Like the cationic inulin polymers, the galactomannans, preferably galactomannans from the aforementioned sources, may be cationically modified by reacting the hydroxyl groups of the galactomannan polymers with reactive quaternary ammonium compounds. Suitable quaternary ammonium compounds are preferably compounds of the following formula

N⁺(R¹R²R³R⁴)X⁻

wherein R¹, R² and R³ are methyl or ethyl groups and R⁴ is an epoxy-R⁵ or a halohydrin group Y—CH₂—CH(OH)—R⁵—, wherein R⁵ is a C₁-C₃ alkylene group, Y is a halide and X is an anion such as Cl−, Br−, I− or HSO4−. Particularly suitable cationic galactomannan polymers within the meaning of the present disclosure correspond to the formula

R—O—CH₂—CH(OH)—R⁵—N⁺(R¹R²R³)X⁻,

wherein R is the respective galactomannan and the other radicals have the same meaning as above.

In a particularly preferred embodiment, the compositions as contemplated herein therefore include cationic galactomannan polymers which have been cationically modified with cationic hydroxy C₁-C₃ alkyltrialkylammonium groups, in particular with hydroxypropyltrimethylammonium groups. Within this embodiment, galactomannan polymers which have been cationically modified with cationic hydroxy C₁-C₃ alkyltrialkylammonium groups, in particular with hydroxypropyltrimethylammonium groups, and which are derived from guar gum and/or from cassia gum are particularly preferred.

In a particularly preferred embodiment, the cosmetic compositions as contemplated herein include, as cationic polymer, e) at least one cationic polysaccharide polymer e) from natural sources, preferably one of guar, a cationic polymer obtainable from cassia and/or inulin and, more preferably, one or more of the compounds known under the INCI names Guar Hydroxypropyltrimonium Chloride, Hydroxypropyl Guar Hydroxypropyltrimonium Chloride, Hydroxypropyl Trimonium Inulin and/or Cassia Hydroxypropyltrimonium Chloride.

The cationic polymer known under the INCI designation Hydroxypropyl Trimonium Inulin is particularly preferred.

The present disclosure further concerns a solid cosmetic composition as described above, including—based on the total weight of the cosmetic composition—from about 0.01 to about 5.00% by weight of at least one oil, fat and/or wax component f), preferably a naturally occurring oil, fat or wax.

These are caring substances which help to keep both the skin and hair structure healthy. The defined concentration range allows this care effect to be used but at the same time prevents noticeable greasiness after application of an appropriate composition. Naturally occurring raw materials have the advantage that they regenerate and are therefore sustainable. This aspect is also becoming increasingly important for many users.

It was discovered that vegetable butters with a melting range of from about 20° C. to about 35° C. are particularly suitable for incorporation into cosmetic compositions as contemplated herein.

Accordingly, vegetable butters with a melting point in the range of from about 20° C. to about 35° C. are particularly preferred as, for example, shea butter (INCI designation: Butyrospermum parkii (Shea) Butter), mango butter (INCI designation): Mangifera indica (Mango) Seed Butter), murumuru butter (INCI designation: Astrocaryum murumuru Seed Butter), cocoa butter (INCI designation: Theobroma cacao (Cocoa) Seed Butter) and/or cupuacu butter (INCI designation: Theobroma grandiflorum Seed Butter).

Cupuacu butter (INCI name: Theobroma grandiflorum Seed Butter) and/or shea butter (INCI name: Butyrospermum parkii (Shea) Butter) are particularly preferred and shea butter (INCI name: Butyrospermum parkii (Shea) Butter) is particularly preferred.

The at least one vegetable butter (preferably cupuacu butter and/or shea butter, in particular shea butter) is used in the cosmetic compositions as contemplated herein preferably in a proportion by weight of from about 0.01 to about 10.00% by weight, more preferably of from about 0.05 to about 5% by weight, particularly preferably of from about 0.10 to about 1% by weight of the total weight of the compositions.

Oils suitable as contemplated herein are preferably perfume oils and/or vegetable triglyceride oils, such as coconut oil, (sweet) almond oil, walnut oil, peach kernel oil, apricot kernel oil, avocado oil, tea tree oil, soybean oil, cotton seed oil, sesame oil, sunflower oil, tsubaki oil, evening primrose oil, rice bran oil, palm oil, Palm kernel oil, mango kernel oil, cranberry oil, sea buckthorn oil, meadow foam herb oil, thistle oil, macadamia nut oil, grape seed oil, amaranth seed oil, argan oil, bamboo oil, olive oil, wheat germ oil, pumpkin seed oil, mallow oil, hazelnut oil, safflower oil, corn oil, olive oil, rapeseed oil, canola oil, sasanqua oil, jojoba oil, rambutan oil, manila oil and/or quinoa oil.

Especially preferred are argan oil, jojoba oil, marula oil, macadamia nut oil, pumpkin seed oil, amaranth seed oil, quinoa oil, soybean oil, cotton seed oil, sunflower oil, palm oil, palm kernel oil, linseed oil, almond oil, corn oil, olive oil, rapeseed oil, sesame oil, soybean oil, safflower oil, wheat germ oil, peach kernel oil, cranberry oil, sea buckthorn oil and/or coconut oil.

Especially preferred are argan oil, jojoba oil, marula oil, almond oil, olive oil, coconut oil and/or sunflower oil.

The oil(s) may be used in the compositions as contemplated herein preferably in a proportion by weight from about 0.01 to about 5%, more preferably from about 0.05 to about 4%, particularly preferably from about 0.10 to about 3%, of the total weight of the compositions.

The present disclosure further concerns a solid cosmetic composition as described above, including—based on the total weight of the cosmetic composition—from about 0.001 to about 0.01% by weight of at least one bitter substance g), preferably a compound known under the INCI designation Denatonium Benzoate.

A bittern is particularly important in cosmetics, household products, etc. which are packaged in such a way that their shape, color, haptics, etc. appeal to small children or babies and encourage them to play, although swallowing could also occur. A bitter substance prevents this. Denatonium benzoate is an extremely strong bittern and is therefore particularly effective even at very low concentrations. It is also not associated with any known harmful effects.

In addition to the ingredients described above, the cosmetic compositions as contemplated herein may include at least one active substance which is advantageously selected from the group comprising plant extracts, humectants, protein hydrolysates, perfumes, UV filters, structurants such as maleic acid, dyes for coloring the composition, active ingredients such as bisabolol and/or allantoin, antioxidants, preservatives such as sodium benzoate or salicylic acid, additional viscosity regulators such as salts (NaCl) or polymers, and pH adjusters such as α and β hydroxycarboxylic acids such as citric acid, lactic acid, malic acid, glycolic acid, and/or bases such as alkanolamines and/or sodium hydroxide.

By suitable plant extracts is meant extracts that may be produced from all parts of a plant. Usually these extracts are produced by extracting the whole plant. However, in some cases it may be preferable to produce extracts exclusively from the flowers and/or leaves of the plant.

Particularly suitable are extracts of Paeonia lactiflora, Rosa damascena flower, Malus domestica fruit, Argania spinosa shell powder, Laminaria saccharina, Cannabis sativa, green tea, oak bark, nettle, Hamamelis, hops, chamomile, burdock root, horsetail, hawthorn, lime blossom, Litchi, almond, Aloe vera, spruce needle, horse chestnut, sandalwood, juniper, coconut, mango, apricot, lime, wheat, kiwi, melon, orange, grapefruit, sage, rosemary, birch, mallow, cuckooflower, thyme, yarrow, thyme, lemon balm, cowslip, marshmallow, ginseng, ginger root, Echinacea purpurea, Olea europea, Boerhavia diffusa roots, Foeniculum vulgare and Apium graveolens.

Particularly preferred for use in the compositions as contemplated herein are the extracts from Paeonia lactiflora, Rosa damascena flower, Malus domestica fruit, Argania spinosa shell powder, Laminaria saccharina, Cannabis sativa, green tea, nettle, witch hazel, chamomile, Aloe vera, ginseng, Echinacea purpurea, Olea europea and/or Boerhavia diffusa roots.

Water, alcohols and mixtures thereof may be used as extraction agents for the production of the above plant extracts. Among the alcohols, lower alcohols such as ethanol and isopropanol, but in particular polyhydric alcohols such as ethylene glycol and propylene glycol, both as the sole extracting agent and in admixture with water, are preferred. Plant extracts based on water/propylene glycol in a ratio of from about 1:10 to about 10:1 have proved to be particularly suitable.

The plant extracts may be used both in pure and diluted form. If they are used in diluted form, they usually include approx. 2-80% by weight of active substance and the extraction agent or mixture of extraction agents used in their extraction as solvent. The plant extracts may be used in the hair treatment preparations as contemplated herein (based on the total weight of the preparations) preferably in an amount from about 0.01 to about 10% by weight, more preferably from about 0.05 to about 7.5% by weight and in particular from about 0.1 to about 5% by weight.

The present disclosure further concerns a solid cosmetic composition as described above for the cleansing and/or care of human skin and/or human hair.

It is advantageous and simple for the user to be able to use the same product for skin and hair. A composition which is suitable for both due to its formulation and packaging may therefore be considered as beneficial.

The present disclosure further concerns a solid cosmetic composition as described above in the form of a powder which dissolves and foams in contact with water.

A powder form is good, namely particularly fast soluble. This is in line with the spirit of the times, but at the same time it is associated with a small “wellness experience” during application, as the use of the powder creates a new and different feeling of application from previous experiences, which emphasizes the playful side of body care.

The present disclosure also relates to a solid cosmetic composition as described above in the form of a pressed or molded body, which has a density in the range of from about 0.6 g/cm³ to about 1.1 g/cm³, and which dissolves and foams in contact with water.

A pressed body corresponds in its appearance most closely to a classic bar of soap, so that it may be preferred by more traditional, less exploratory users.

The present disclosure also relates to a solid cosmetic composition as described above in the form of a porous body which has a density in the range of from about 0.2 g/cm³ to about 0.9 g/cm³ and which dissolves and foams in contact with water.

A porous body feels interesting, which enriches the user sensory, and also dissolves well and quickly due to the large surface, which may save time but also water.

The present disclosure also relates to a method for preparing a solid cosmetic composition as described above, wherein all ingredients are mixed in a mixing device for cosmetic powder products and sieved if necessary.

This has the advantage of providing a solid cosmetic cleansing composition in powder form, and the sieving ensures a uniform and pleasantly perceived granularity. Another advantage of such a method is that it can be carried out at high temperatures without melting, which saves energy and is very gentle on the ingredients. This is particularly important when largely natural, little processed ingredients are used, which may offer advantages that cannot be achieved with highly processed products.

The present disclosure further concerns a method for preparing a solid cosmetic composition as described above, exemplified by the following steps:
a) mixing of all ingredients in a mixing device for cosmetic powder products,
b) converting the mixture into a suitable form, and
c) pressing the mixture into the desired shape.

The advantage of such a method is that if the advantages explained above are achieved at the same time, a packaging form may be produced which appears relatively traditional and also appeals to more conservative users.

The present disclosure further concerns a method for preparing a solid cosmetic composition as described above, exemplified by the following steps:
a) adding all ingredients into a heatable container,
b) heating the mixture a) at a temperature of up to approx. 75° C. until the ingredients are mixed,
c) extruding the mixture a) from a desired shaping port.

The advantage of such a method is that it is well suited for continuous operation, which is very advantageous for industrial scale production, and that it opens up creative design possibilities through the shaping port.

The present disclosure further concerns a method for preparing a solid cosmetic composition as described above, exemplified by the following steps:
a) adding all ingredients into a heatable container,
b) heating the mixture a) at a temperature of up to approx. 75° C. until the ingredients are mixed,
c) introducing a gas by
   i. introducing air, N₂, N₂O and/or CO₂ at a pressure of from about 2 to about 40 bar, or
   ii. introducing air with a high speed mixer,
d) extruding the fumigated mixture c) from a desired shaping port or into a desired mold,
e) solidification/cooling of the extrudate to the desired shape.

Such a method makes products in the form of solid foams accessible, which are of sensory interest to the user and thus represent enrichment. In addition, the solid foams dissolve well and quickly as they have a large surface area. This may help to save time and water.

The present disclosure further concerns solid cosmetic cleansing compositions available according to the methods described above. Such cleansing compositions offer the advantages described for the respective processes.

The present disclosure further concerns a method for cleansing the human body including the hair, wherein a solid cosmetic composition as described above is moistened with water or soaked or dissolved in water, rubbed between the hands and/or foamed, spread on the body part to be cleansed and rinsed with water.

This method makes the advantages described above tangible for a user and thus represents enrichment not only for personal hygiene and care but also a sensory enrichment.

The present disclosure further concerns the use of a cleansing composition as described above for cleansing and/or care of the human body including the hair.

This application makes the advantages described above perceptible to a user and thus represents enrichment not only for personal hygiene and care but also a sensory enrichment.

As has been shown by employing the previously described embodiments and their advantages, the process and packaging aspects are important for the present disclosure. They are discussed in more detail below.

In a manufacturing process as contemplated herein, for example, all ingredients are placed in a heatable container, such as, on a laboratory scale, in a suitable vessel in a water bath or on a heating plate, on a production scale rather in a closed and pressurizable vessel, and are mixed and heated, in the case of recipes as contemplated herein, for example at about 75° C. until all ingredients are sufficiently mixed. In such a method, various temperature steps may also be carried out. For example, components may be mixed first which are homogeneously miscible even at a relatively low temperature. This may be done at from about 40° C. to about 50° C. It may also be advantageous to mix in certain ingredients at higher temperatures, for example at from about 85° C. to about 90° C. For this purpose, a method as contemplated herein may comprise one or more steps in this temperature range. Afterwards, one or more steps may be carried out at a lower temperature again, wherein further ingredients are mixed in. Typically, the compositions as contemplated herein solidify at about 65° C., so that certain method steps, such as mixing and extruding the finished mixtures, are not reasonably possible below such a temperature level.

A gas may also be introduced into a mixture resulting from a method described above, including gas mixtures such as air, N₂, N₂O and/or CO₂. This may be done in a boiler, for example at from about 200-4000 kPa, or by introducing air, for example, using a high-speed mixer or similar equipment. The resulting mixture may then be discharged via an extruder. The mixture expands if it has been under high pressure, as is the case here, and solidifies, for example, at normal room temperature of from about 18° C. to about 25° C., additionally favored by cooling, which goes hand in hand with the expansion of the contained gas. Otherwise, if it was previously mixed under ambient pressure, the mixture only cools down and solidifies by assuming the ambient temperature or by additionally provided cooling.

The introduction of a gas or gas mixture into the compositions as contemplated herein is accompanied by various advantages. As explained at the beginning, a good dissolving behavior is important for solid cosmetic compositions, in particular for compositions which are also intended to be used in single application portions. In addition to the composition, the dissolution behavior may be influenced by the manufacturing and packaging method. For example, by introducing a gas or gas mixture, the surface area where contact with water may take place may be increased, resulting in faster dissolution and the extremely fine bubble structure already established in the solid composition.

Furthermore, it is important to note that cosmetic products have a lot to do with feeling, fun and emotions. Many people relax while taking care of their bodies and enjoy the pleasant feeling of doing something good for themselves. Especially as many people find their everyday life more and more demanding or stressful, small pleasures and playfulness are an important point where stress can be released from a person and satisfaction can be created. Solid cosmetic preparations with an incorporated gas phase, i.e. to a certain extent solid foams, feel different to conventional products, which are perceived as interesting and pleasant.

Foamed solidified emulsions as such have already been mentioned before, but it should be mentioned here that the consistency of the foamed extrudate and the corresponding production equipment allow the realization of imaginative shapes, as known, for example, from meringue.

Another exciting new form of application of the compositions as contemplated herein, which is associated with a special experience, is packaging in a bag or a glass, where the solid cosmetic composition is in powder form. In addition to the new sensory experience, this is accompanied by the advantage of relatively rapid dissolution due to a high surface area. The packaging materials glass and paper are easily recyclable packaging materials which are popular with environmentally conscious buyers and whose properties therefore fit well with the properties of the solid cosmetic compositions described and claimed here.

It is also possible to fill the solid cosmetic compositions as contemplated herein into a jar, for example, made of glass. Since the firmness of these compositions is in a range that allows an application portion to be removed manually, without a tool, from a crucible intended for multiple applications. If the composition has been foamed in a crucible during its manufacture, this gives a particularly interesting sensation.

After a detailed explanation of the various designs and their respective advantages, the presentation of exemplary compositions and an exemplary manufacturing process follows. Basic exemplary compositions are shown in the following tables 1-4:

TABLE 1

| Ingredient | Z1 | Z2 | Z3 | Z4 | Z5 | Z6 | Z7 | Z8 | Z9 |
|---|---|---|---|---|---|---|---|---|---|
| a): Surfactant, in | 15-50 | 16-49 | 17.5-47.5 | 18-46 | 20-45 | 21-44 | 22.5-42.5 | 23-41 | 25-40 |
| particular anionic | | | | | | | | | |
| surfactant | | | | | | | | | |
| b): Polyhydric C₂—C₄ | 10-60 | 12.5-60 | 15-60 | 17.5-60 | 20-60 | 22.5-60 | 25-60 | 27.5-60 | 30-60 |
| alcohol | | | | | | | | | |
| c): Saturated or | 0.1-10 | 0.25-10 | 0.5-10 | 0.75-10 | 1.0-10 | 1.5-10 | 2.0-10 | 2.5-10 | 3.0-10 |
| unsaturated, branched or | | | | | | | | | |
| unbranched C₈—C₃₀ | | | | | | | | | |
| alcohol; and/or | | | | | | | | | |
| saturated or unsaturated, | | | | | | | | | |
| branched or unbranched | | | | | | | | | |
| C₈—C₃₀ carboxylic acid | | | | | | | | | |
| and/or a salt of a | | | | | | | | | |
| saturated or unsaturated, | | | | | | | | | |
| branched or unbranched | | | | | | | | | |
| C₈—C₃₀ carboxylic acid | | | | | | | | | |
| d): Polysaccharide | 0.1-20 | 0.25-19 | 0.5-17.5 | 0.75--16 | 1.0-15 | 1.25-14 | 1.5-13.5 | 1.75-12 | 2.0-10 |
| Water and, if necessary, | ad | ad | ad | ad | ad | ad | ad | ad | ad |
| other auxiliary materials | 100 | 100 | 100 | 100 | 100 | 100 | 100 | 100 | 100 |
| and additives | | | | | | | | | |

TABLE 2

| Ingredient | Z11 | Z11 | Z12 | Z13 | Z14 | Z15 | Z16 | Z17 | Z18 |
|---|---|---|---|---|---|---|---|---|---|
| Isethionate surfactant | 15-50 | 16-49 | 17.5-47.5 | 18-46 | 20-45 | 21-44 | 22.5-42.5 | 23-41 | 25-40 |
| and/or taurate surfactant | | | | | | | | | |
| 1,2-propylene glycol, | 10-60 | 12.5-60 | 15-60 | 17.5-60 | 20-60 | 22.5-60 | 25-60 | 27.5-60 | 30-60 |
| 1,3-butylene glycol, | | | | | | | | | |
| dipropylene glycol, | | | | | | | | | |
| glycerol and/or | | | | | | | | | |
| diglycerol | | | | | | | | | |
| Coco acids, lauric acid, | 0.1-10 | 0.25-10 | 0.5-10 | 0.75-10 | 1.0-10 | 1.5-10 | 2.0-10 | 2.5-10 | 3.0-10 |
| myristic acid, palmitic | | | | | | | | | |
| acid, stearic acid, | | | | | | | | | |
| behenic acid, oleic acid | | | | | | | | | |
| and/or their salts | | | | | | | | | |
| Starch fractions from | 0.1-20 | 0.25-19 | 0.5-17.5 | 0.75--16 | 1.0-15 | 1.25-14 | 1.5-13.5 | 1.75-12 | 2.0-10 |
| maize, potatoes, rice, | | | | | | | | | |
| wheat and/or tapioca | | | | | | | | | |
| and/or derivatives of | | | | | | | | | |
| starches such as | | | | | | | | | |
| amylose, amylopectin, | | | | | | | | | |
| dextrin, and/or | | | | | | | | | |
| cellulose and/or | | | | | | | | | |
| cellulose derivatives | | | | | | | | | |
| such as alkyl cellulose, | | | | | | | | | |
| carboxyalkyl cellulose, | | | | | | | | | |
| hydroxyalkyl cellulose. | | | | | | | | | |
| Water and, if necessary, | ad | ad | ad | ad | ad | ad | ad | ad | ad |
| other auxiliary materials | 100 | 100 | 100 | 100 | 100 | 100 | 100 | 100 | 100 |
| and additives | | | | | | | | | |

TABLE 3

| Ingredient | Z19 | Z20 | Z21 | Z22 | Z23 | Z24 | Z25 | Z26 | Z27 |
|---|---|---|---|---|---|---|---|---|---|
| Isethionate surfactant | 10-40 | 11-37.5 | 12.5-35 | 14-34 | 15-32.5 | 16-31 | 17.5-30 | 18-27.5 | 20-25 |
| according to formula (I) | | | | | | | | | |
| Taurate surfactant | 1.0-10 | 1.5-9.5 | 2.0-9.0 | 2.25-8.5 | 2.5-8.0 | 2.75-7.5 | 3.0-7.0 | 3.0-7.0 | 3.0-7.0 |
| according to formula (II) | | | | | | | | | |
| Glycerin | 10-60 | 12.5-60 | 15-60 | 17.5-60 | 20-60 | 22.5-60 | 25-60 | 27.5-60 | 30-60 |
| Coco acids, stearic acid | 0.1-10 | 0.25-10 | 0.5-10 | 0.75-10 | 1.0-10 | 1.5-10 | 2.0-10 | 2.5-10 | 3.0-10 |
| and/or their salts | | | | | | | | | |
| Maize starch | 1.0-15 | 1.1-14 | 1.25-13.5 | 1.4-13 | 1.5-12.5 | 1.75-12 | 2.0-11.5 | 2.5-11 | 3.0-10 |
| Maltodextrin | 0.1-10 | 0.2-9.0 | 0.3-8.5 | 0.4-8.0 | 0.5-7.5 | 0.6-7.0 | 0.7-6.5 | 0.75-6.0 | 0.75-5.5 |
| Water and, if necessary, | ad | ad | ad | ad | ad | ad | ad | ad | ad |
| other auxiliary materials | 100 | 100 | 100 | 100 | 100 | 100 | 100 | 100 | 100 |
| and additives | | | | | | | | | |

TABLE 4

| Ingredient | Z28 | Z29 | Z30 | Z31 | Z32 | Z33 | Z34 |
|---|---|---|---|---|---|---|---|
| a): Surfactant, in particular anionic | 15-50 | 15-50 | 15-50 | 15-50 | 15-50 | 15-50 | 15-50 |
| surfactant | | | | | | | |
| b): Polyhydric C₂—C₄ alcohol | 10-60 | 10-60 | 10-60 | 10-60 | 10-60 | 10-60 | 10-60 |
| c): Saturated or unsaturated, | 0.1-10 | 0.1-10 | 0.1-10 | 0.1-10 | 0.1-10 | 0.1-10 | 0.1-10 |
| branched or unbranched C₈—C₃₀ | | | | | | | |
| alcohol; and/or | | | | | | | |
| saturated or unsaturated, branched | | | | | | | |
| or unbranched C₈—C₃₀ carboxylic | | | | | | | |
| acid and/or a salt of a saturated or | | | | | | | |
| unsaturated, branched or | | | | | | | |
| unbranched C₈—C₃₀ carboxylic | | | | | | | |
| acid | | | | | | | |
| d): Polysaccharide | 0.1-20 | 0.1-20 | 0.1-20 | 0.1-20 | 0.1-20 | 0.1-20 | 0.1-20 |
| e): Cationic polymer | 0.01-5.0 | | | 0.01-5.0 | 0.01-5.0 | | 0.01-5.0 |
| f): Oil, fat and/or wax component | | 0.01-5.0 | | 0.01-5.0 | | 0.01-5.0 | 0.01-5.0 |
| g): Bittern | | | 0.001-0.01 | | 0.001-0.01 | 0.001-0.01 | 0.001-0.01 |
| Water and, if necessary, other | ad | ad | ad | ad | ad | ad | ad |
| auxiliary materials and additives | 100 | 100 | 100 | 100 | 100 | 100 | 100 |

A detailed exemplary composition is shown in the following Table 5:

TABLE 5

| | | active ingredients contained | |
|---|---|---|---|
| Group | Ingredients | therein (INCI) | Z36 |
| 1 | Pureact ® WS Cone | Aqua (water), sodium | 11.15 |
| | | methyl cocoyl taurate, | |
| | | coconut acid, sodium | |
| | | chloride | |
| 1 | City water | Aqua (water) | 0.25 |
| 1 | Glycerin 99.5% | Glycerin | 41 |
| 1 | Sodium stearate OP-100V | Sodium stearate | 6 |
| 1 | Citric acid | Citric acid | 2 |
| 2 | ISELUX ® | Sodium lauroyl methyl | 2 |
| | | isethionate, sodium methyl | |
| | | isethionate | |
| 2 | Maltodextrin DE15 | Maltodextrin | 1 |
| 3a | Pureact I-80P ® | Sodium cocoyl isethionate | 25.5 |
| 3b | Maisita 9040 ® | Zea mays (corn) starch | 5 |
| 4 | Avocado oil, cold- | Persea gratissima (avocado) | 2 |
| | pressed | oil, ascorbyl palmitate | |
| 4 | Preservative | Phenoxyethanol | 1 |
| 4 | Fragrance | Perfume (fragrance) | 1 |

The exemplary method was carried out as follows:

The composition shown in detail in Table 5 was produced as follows: The ingredients were used in the proportions shown in Table 2 above. Pureact WS Conc was heated in a drum at 40° C. to 50° C. to melt and mix. After mixing to homogeneity, the other ingredients of Group 1 (see Table 2) were added. Mixing to homogeneity was repeated and then the temperature was increased to 85° C. to 90° C. At this temperature the ingredients of Group 2 (see Table 2) were added and mixed in until homogeneity was achieved. This was then repeated with the ingredients of Group 3 (3a and 3b, see Table 2). The ingredients of Group 4 (see Table 2) were homogeneously mixed together and also added to the previously prepared mixture and blended in until homogeneity was achieved. For the further steps, the temperature was no longer actively maintained at 85° C. to 90° C. but it was only ensured that it did not drop to 70° C. or less. Thereupon all ingredients of Group 5 (see Table 2) were added sequentially and mixed in until homogeneity was achieved and the ingredient of Group 6 (see Table 2) was added and quickly mixed in until homogeneity was achieved. Finally the mixture was kept at a temperature above 70° C. for filling or packaging.

This was followed by the introduction of a gas selected from air, N₂, N₂O and/or CO₂ at a pressure of 2 to 40 bar or alternatively the introduction of air with a high speed mixer, extrusion of the gassed mixture from a desired shaping port or into a desired mold and solidification/cooling of the extrudate in the desired shape and removal of the extrudate from the mould or cutting and portioning of the extrudate. The cosmetic compositions obtained had densities ranging from 0.2 g/cm³ to 1.2 g/cm³.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Solid cosmetic composition comprising—based on the total weight of the cosmetic composition—
a) from about 15.0 to about 50.0% by weight of at least one surfactant,
b) from about 10.0 to about 60.0% by weight of at least one polyhydric C₂-C₆ alcohol,
c) from about 0.1 to about 10.0% by weight of at least one saturated or unsaturated, branched or unbranched C₈-C₃₀ alcohol and/or a saturated or unsaturated, branched or unbranched C₈-C₃₀ carboxylic acid and/or a salt of a saturated or unsaturated, branched or unbranched C₈-C₃₀ carboxylic acid, and
d) from about 0.1 to about 20% by weight of at least one polysaccharide selected from
starch fractions and/or derivatives of starches, and/or
cellulose and/or cellulose derivatives.

2. Solid cosmetic composition according to claim 1, comprising—based on the total weight of the cosmetic composition—
i. from about 10.0 to about 40% by weight of at least one anionic isethionate surfactant, and
ii. from about 1.0 to about 10% by weight of at least one anionic taurate surfactant.

3. Solid cosmetic composition according to claim 1, comprising as polyhydric alcohol b) 1,2-propylene glycol, 1,3-butylene glycol, dipropylene glycol, glycerol and/or diglycerol.

4. Solid cosmetic composition according to claim 1, comprising as polyhydric alcohol b) glycerol.

5. Solid cosmetic composition according to claim 1, comprising as component c) saturated or unsaturated, branched or unbranched C₈-C₃₀ carboxylic acids and/or their salts.

6. Solid cosmetic composition according to claim 1, comprising as component c) saturated or unsaturated, branched or unbranched C₁₀-C₂₂ carboxylic acids and/or their salts.

7. Solid cosmetic composition according to claim 1, comprising as component c) coco acids, lauric acid, myristic acid, palmitic acid, stearic acid, behenic acid, oleic acid and mixtures thereof and/or the salts of these acids.

8. Solid cosmetic composition according to claim 1, comprising as polysaccharide d)
i. starch fractions from corn, potatoes, rice, wheat and/or tapioca, and/or derivatives thereof,
ii. cellulose and/or cellulose derivatives.

9. Solid cosmetic composition according to claim 1 in a form chosen from
a) a powder which dissolves and foams when in contact with water,
b) a pressed or shaped body, which has a density in the range from about 0.6 g/cm³ to about 1.1 g/cm³, and which dissolves and foams in contact with water, or
c) a porous body which has a density in the range from about 0.2 g/cm³ to about 0.9 g/cm³ and which dissolves and foams in contact with water.

10. Method for the preparation of a solid cosmetic composition according to claim 9, wherein all the ingredients are mixed in a mixing device for cosmetic powder products and optionally sieved.

11. Method for the preparation of a solid cosmetic composition according to claim 9, comprising:
a) mixing all ingredients in a mixing device for powdered cosmetic products,
b) converting the mixture into a suitable form, and
c) pressing the mixture into the desired shape.

12. Method for the preparation of a solid cosmetic composition according to claim 9, comprising:
a) adding all ingredients into a heatable container,
b) heating the mixture a) at a temperature of up to approx. 75° C. until the ingredients are mixed,
c) extruding the mixture a) from a desired shaping port.

13. Method for the preparation of a solid cosmetic composition according to claim 9, comprising:
a) adding all ingredients into a heatable container,
b) heating the mixture a) at a temperature of up to approx. 75° C. until the ingredients are mixed,
c) introducing a gas by
i. introducing air, N₂, N₂O and/or CO₂ at a pressure of from about 2 to about 40 bar, or
ii. introducing air with a high speed mixer,
d) extruding the gassed mixture c) from a desired shaping port or into a desired shape,
e) solidification/cooling of the extrudate to the desired shape.

14. Solid cosmetic composition comprising—based on the total weight of the cosmetic composition—
a) from about 25.0 to about 40.0% by weight of at least one surfactant,
b) from about 30.0 to about 60% by weight of glycerol,
c) from about 3.0 to about 10% by weight of at least one saturated or unsaturated, branched or unbranched C₁₀-C₂₂ carboxylic acid and/or their salts, and
d) from about 0.1 to about 20% by weight of at least one polysaccharide selected from
starch fractions from corn, potatoes, rice, wheat and/or tapioca, and/or derivatives thereof, and/or
cellulose and/or cellulose derivatives.
